(19) United States
(12) Patent Application Publication
(10) Pub. No.: US 2025/0255732 A1
(43) Pub. Date: Aug. 14, 2025

(54) IMPROVEMENTS IN AND RELATING TO SURGICAL COMPONENT MOUNTING SYSTEMS

(71) Applicant: DEPUY IRELAND UNLIMITED COMPANY, RINGASKIDDY (IE)

(72) Inventors: AMY KINBRUM, YORK (GB); ELLIOT DREDGE, LEEDS (GB)

(21) Appl. No.: 18/855,789

(22) PCT Filed: Apr. 11, 2023
(86) PCT No.: PCT/EP2023/059407

§ 371 Date: Oct. 10, 2024

(30) Foreign Application Priority Data
Apr. 11, 2022 (GB) 2205303.7

(51) Int. Cl. A61F 2/46; A61B 17/92; A61F 2/30

(52) U.S. Cl. CPC A61F 2/4607; A61B 17/92; A61F 2/4609; A61F 2002/30604; A61F 2002/30617; A61F 2002/30716

(57) ABSTRACT

A surgical component mounting system is provided that can include a surgical instrument (5) and an intermediate component (20). The surgical instrument can have a proximal end and a distal end (7) that can provide a first part (9) of a mounting. The intermediate component can have a proximal end that can provide a second part (22) of the mounting and a distal end (35) that can be provided with a first part (36, 38a, 38b) of a second mounting such that the first part of the mounting and the second part of the mounting are detachable.

The present application claims priority to GB Patent Application No. 2205303.7 which was filed on Apr. 11, 2022 and is incorporated in its entirety by reference herein.

### TECHNICAL FIELD

The present disclosure relates generally to orthopaedic devices, kits including such devices and methods of using such devices and more particularly to orthopaedic impaction devices, kits including such devices and methods of using such devices. The disclosure also has wider relevance in the mounting of other surgical instruments.

### BACKGROUND

Joint arthroplasty is a well-known surgical procedure by which a diseased and/or damaged natural joint is replaced by a prosthetic joint. Examples include knee prosthesis and hip implants. During insertion and positioning of, for example a femoral implant into a femur, an impaction tool is often engaged with the implant and then impacted to move the implant as desired.

### SUMMARY

According to a first aspect of the disclosure there is provided a surgical component mounting system, the system comprising:
a surgical instrument, the surgical instrument having a proximal end and a distal end, the distal end of the surgical instrument providing a first part of a mounting;
an intermediate component, the intermediate component having a proximal end, the proximal end of the intermediate component providing a second part of the mounting, the intermediate component having a distal end, the distal end of the intermediate component being provided with a first part of a second mounting; wherein the first part of the mounting and the second part of the mounting are detachable.

The intermediate component may be a single surgical occasion use component. The surgical instrument may be a multiple surgical occasion use instrument.

The intermediate component may be a used once and then discarded. The surgical instrument may be used once and then sterilised and reused, for instance with a new intermediate component.

The intermediate component may be provided with one or more characteristics which inhibit the intermediate component being a multiple surgical occasion use component.

The one or more characteristics may be susceptible to change under one or more conditions. The one or more conditions may be one or more conditions experienced by the intermediate component if prepared for reuse. The one or more conditions may be a washing temperature and/or exposure to one or more chemicals and/or exposure to a disinfecting temperature and/or exposure to a sterilising temperature.

The one or more characteristics may be one or more visual indicia. The one or more visual indicia may be the presence of a visual indicia and/or the absence of a visual indicia. The one or more visual indicia may be the presence of a colour. The one or more visual indicia may be a change in colour from a first colour to a second colour. The one or more indicia may be a warning.

The one or more characteristics may be a changeable part of the intermediate component. The one or more characteristics may be a part of the intermediate component that changes profile or shape, for instance to prevent the intermediate component being connected to the surgical component. The one or more characteristics may be a part of the intermediate component that ceases to be present, for instance the retention elements no longer being present. The one or more characteristics may be a part of the intermediate component that cease to function, for instance to retain the intermediate component on the surgical instrument.

The detachable mounting may be adapted to, in an engaged state, restrain axial movement of the surgical component and the intermediate component relative to one another in one or both axial directions. The detachable mounting may be adapted to be sufficient to retain the intermediate component on the surgical instrument or vice versa, for instance during manipulation of the surgical instrument. The detachable mounting may be adapted to provide restraint which is insufficient to stop a user pulling the intermediate component off the surgical instrument.

In a potentially alternative form, the detachable mounting may be adapted to provide, in an engaged state, the first part of the mounting and the second part of the mounting to restrain axial movement of the surgical component and the intermediate component relative to one another, further towards one another. In the alternative form, in the engaged state the detachable mounting may be adapted not to restrain axial movement of the surgical component and the intermediate component relative to one another, away from each other.

The intermediate component may be a cap element. The intermediate component may provide a body part. The intermediate component may provide a recess, such as a bore, for instance a closed end bore, potentially in the body part. The recess or bore may have a longitudinal axis, for instance aligned with the longitudinal axis of the intermediate component and/or the longitudinal axis of the surgical instrument. The recess or bore may be defined by one or more of a side wall, base wall and a transition surface, such as a fillet, potentially provided between the side wall and the base wall. The base wall may be substantially perpendicular to the longitudinal axis of the recess or bore or to the longitudinal axis of the intermediate component.

The recess or bore may be provided with a mouth, preferably the mouth is outwardly flared or tapered.

The recess or bore may have a cross-section considered perpendicular to the longitudinal axis of the recess or bore and/or of the intermediate element.

The intermediate component, for instance a body part, may provide an external wall. The intermediate component, for instance a body part, may be a right cylinder. One or more locations on the outside of the intermediate component, such as body part, may be provided with grooves and ridges or other recess/protrusion combinations, for instance to assist with grip.

The recess or bore may be provided with one or more retention elements. The recess or bore may be provided with one or more projections extending inwardly, for instance radially inwardly. The projection may be in the form of a rib or ridge, for instance a rounded cross-section rib or ridge.

The retention element[s], such as the projection[s], may extend around the internal perimeter of the recess or bore at a location. The retention element[s] may be provided at a constant axial distance from the proximal end of the intermediate element and/or from the distal end of the intermediate element. The retention element[s] may be provided at a constant depth relative to a mouth of the recess or bore and/or at a constant height relative to the base wall of the recess or bore.

The distal end of the intermediate element may provide a first part of the second mounting which includes one or more projections. One or more or all, of the projections may extend in a distal direction, for instance parallel to the longitudinal axis of the recess or bore and/or parallel to the longitudinal axis of the intermediate element. The first part of the second mounting may include a primary projection and one or two or more secondary projections.

The primary projection may extend along the longitudinal axis of the recess or bore and/or along the longitudinal axis of the intermediate element. The primary projection may be provided between two or more secondary projections, for instance between a pair of secondary projections. One or more or all of the secondary projections may extend parallel to the longitudinal axis of the recess or bore and/or parallel to the longitudinal axis of the intermediate element. One or two or all, of the secondary projections may be tines.

Two or more projections may be separated from one another by a gap, for instance a recess. The gap or recess may be curved, for instance with a half-circle cross-section. The gap or recess may have a reduced axial extent in the distal direction compared with the projections. The primary projection may be separated from two secondary projections by a gap or recess in each case.

The first part of the second mounting may have a greater extent perpendicular to the longitudinal axis of the recess or bore and/or to the longitudinal axis of the intermediate element in a first direction compared with the extent in a second direction. The second direction may be perpendicular to the first direction. The first direction may be the medial and/or lateral direction and/or the second direction may be the anterior and/or posterior direction. The first part of the second mounting may have a greater extent perpendicular to the longitudinal axis of the recess or bore and/or to the longitudinal axis of the intermediate element in the medial and/or lateral direction compared with the extent in the anterior and/or posterior direction. The medial and/or lateral direction extent may be greater than 60% of the maximum radial extend of the intermediate component. The anterior and/or posterior direction extent may be less than 40% of the maximum radial extent of the intermediate component.

In an embodiment, a first part of the recess or bore may be provided with a cross-section which decreases away from the proximal end. The first part may define a mouth of the recess or bore. A second part of the recess or bore may be provided with a cross-section which decreases further away from the proximal end and then increases again still further away from the proximal end. The second part may define one or more retention elements. A third part of the recess or bore may be provided with a cross-section which decreases, for instance a slight taper. The first part may form less than 20% of the depth of the recess or bore. The second part may form less than 20% of the depth of the recess or bore. The third part may provide at least 65% of the depth of the recess or bore.

In an embodiment, the recess or bore is enclosed by a body part and the recess or bore has a maximum radial extent which is at least 65% of the maximum radial extent of the body part. The recess or bore may have a circular cross-section and/or the body part may have a circular cross-section.

In an embodiment, the recess or bore may be provided in a body part and the recess or bore may have a maximum depth which is less than 70% of the axial length of the intermediate component from proximal end to distal end.

In a second embodiment, a first part of the recess or bore may be provided with a cross-section which decreases away from the proximal end. The first part may define a mouth of the recess or bore. A second part of the recess or bore may be provided with a cross-section which decreases further away from the proximal end and then increases again still further away from the proximal end. The second part may define one or more retention elements. A third part of the recess or bore may be provided with a cross-section which decreases, for instance a slight taper. The first part may form less than 20% of the depth of the recess or bore. The second part may form less than 20% of the depth of the recess or bore. The third part may provide at least 65% of the depth of the recess or bore.

In a second embodiment, the recess or bore is enclosed by a body part and the recess or bore has a maximum radial extent which is at least 35% of the maximum radial extent of the body part. The recess or bore may have a circular cross-section and/or the body part may have a circular cross-section.

In a second embodiment, the recess or bore may be provided in a body part and the recess or bore may have a maximum depth which is less than 70% of the axial length of the intermediate component from proximal end to distal end.

In a third embodiment, a first part of the recess or bore may be provided with a cross-section which decreases away from the proximal end. The first part may define a mouth of the recess or bore. No second part of the recess or bore may be provided which has a cross-section which decreases further away from the proximal end and then increases again still further away from the proximal end. No second part may be provided which may define one or more retention elements. A second part of the recess or bore may be provided with a cross-section which decreases, for instance a slight taper. The first part may form less than 20% of the depth of the recess or bore. The second part may form at least 75% of the depth of the recess or bore.

In a third embodiment, the recess or bore is enclosed by a body part and the recess or bore has a maximum radial extent which is at least 35% of the maximum radial extent of the body part. The recess or bore may have a circular cross-section and/or the body part may have a circular cross-section.

In a third embodiment, the recess or bore may be provided in a body part and the recess or bore may have a maximum depth which is less than 70% of the axial length of the intermediate component from proximal end to distal end.

In a third embodiment, an interference fit may be employed between the first part of the mounting and the second part of the mounting.

In a fourth embodiment, the mounting may be a bayonet type mounting. The female part with one or more radially extending lugs or pins, for instance extending inward, may be provided on the intermediate component. The male part with one or more slots, for instance recessed in the outer surface, for receiving one or more lugs or pins may be provided on the surgical instrument.

In a fifth embodiment, the mounting may be a threaded type mounting. The male part with the external screw thread may be provided on the surgical instrument. The female part with the internal screw thread may be provided on the intermediate component.

In a sixth embodiment, the mounting may be a complimentary surface type mounting. For instance, a complementary protrusion profile may be provided on one of the surgical instrument and intermediate component and a complimentary recess profile therefore may be provided on the other of the surgical instrument and intermediate component. The complimentary protrusion profile may be a cross shaped protrusion, for instance with one or more or all side walls thereof substantially parallel with the longitudinal axis of the surgical instrument or intermediate component the profile is provided on. The complimentary recess profile may be a cross shaped recess, for instance with one or more or all side walls thereof substantially parallel with the longitudinal axis of the surgical instrument or intermediate component the profile is provided on.

The surgical instrument may be an orthopaedic instrument. The surgical instrument may be an impactor instrument. The surgical instrument may be used to apply axial force or substantially axial force, for instance to an implant and/or a further surgical instrument. The surgical instrument may be used to apply a lateral force, for instance medially and/or laterally, in place of or as well as an axial force or substantially axial force.

The axial force may variable, for instance due to another instrument impacting the surgical instrument. The force, such as axial force, may be applied to the proximal end of the surgical instrument. The another instrument may apply the force to the surgical instrument, then through the intermediate component, then to the implant. The another instrument may apply the force to the surgical instrument, then through the intermediate component, then through the further surgical instrument, then to the implant.

The impactor instrument may be for an implant. The implant may be a femoral implant. The implant may be an acetabular cup. The implant may be an acetabular shell.

The distal end of the surgical instrument may be configured to engage with the intermediate component by means of the mounting, such as the proximal end of the intermediate component.

The distal end of the surgical instrument may comprise a stem. The stem may have the same cross-sectional profile as other adjacent sections of the surgical instrument. The stem may be a shaft, for instance with a circular cross-section. The stem may have a longitudinal axis, for instance aligned with the longitudinal axis of at least the adjacent part of the surgical instrument and/or, in use, with that of the intermediate component. The stem may be defined by one or more of a side wall, an end wall and a transition surface, such as a fillet, potentially provided between the side wall and the end wall. The end wall may be substantially perpendicular to the longitudinal axis of the stem and/or to the longitudinal axis of the surgical instrument and/or, in use, to the intermediate component.

The stem may have a cross-section considered perpendicular to the longitudinal axis of the stem and/or adjacent section of the surgical instrument and/or, in use, to the intermediate element.

The stem may provide an external wall. The stem may be a right cylinder.

The stem may be provided with one or more stem retention elements. The stem may be provided with one or more recesses extending inwardly, for instance radially inwardly. The recesses may be in the form of a groove, for instance a rounded groove.

The stem retention element[s], such as the recess [es], may extend around the external perimeter of the stem. The stem retention element[s] may be provided at a constant axial distance from the distal end of the stem.

The stem retention element[s] may be provided at a consistent distance from the distal end of the surgical component.

The stem and/or adjacent section of the surgical instrument may have a longitudinal axis which is inclined or off-set relative to the longitudinal axis of another part of the surgical instrument. The proximal end of the surgical instrument may be offset from the longitudinal axis of the stem and/or adjacent section.

At least a part of the external profile of the stem may be complimentary to at least a part of the internal profile of the intermediate component. The end face of the stem may be complimentary to the base wall of the recess or bore in the intermediate component. The side wall of the stem may be at least partially complimentary to the side wall of the bore or recess, at least partially.

At least a part of the stem retention elements may be complimentary to at least a part of the retention elements of the intermediate component. At least a part of the retention elements of the intermediate component may be received within at least a part of the stem retention components.

The axial distance of the stem retention element[s] may be complimentary to the axial distance of the retention element[s] of the intermediate component from the base wall of the recess or bore.

The mounting may include a male part and a female part. The male part may be provided on the surgical instrument. The female part may be provided on the intermediate component.

The first part of the mounting may be complimentary to the second part of the mounting.

The second mounting may include a male part and a female part. The male part may be provided on the intermediate component. The female part may be provided on the further surgical component and/or implant.

The first part of the second mounting may be complimentary to the second part of the second mounting.

The second part of the mounting may be complimentary to the first part of the mounting. The second part of the mounting may include a recess. The recess may be at least partially complimentary to a projection of the first part of the mounting.

In an embodiment, particularly the section of stem received in the intermediate component, may provide a first part of the stem may be provided with a cross-section which increases away from the distal end. The first part may define an end for the stem. A second part of the stem may be provided with a cross-section which increases, for instance a slight taper. A third part of the stem may be provided with a cross-section which decreases further away from the distal end and then increases again still further away from the distal end. The third part may define one or more stem retention elements. A fourth part of the stem may be provided with a cross-section which increase, for instance a slight taper, or is constant. The first part may form less than 20% of the section of stem received in the intermediate component. The second part may form more than 50% of the section of stem received in the intermediate component. The third part may form less than 20% of the section of stem received in the intermediate component. The fourth part may provide less than 20% of the section of stem received in the intermediate component.

In a second embodiment, particularly the section of stem received in the intermediate component, may provide a first part of the stem may be provided with a cross-section which increases away from the distal end. The first part may define an end for the stem. A second part of the stem may be provided with a cross-section which increases, for instance a slight taper. A third part of the stem may be provided with a cross-section which decreases further away from the distal end and then increases again still further away from the distal end. The third part may define one or more stem retention elements. A fourth part of the stem may be provided intermediate the third part and fifth part that is not received within the intermediate component. The fifth part may be provided with a larger cross-section than the first part and/or second part and/or third part and/or fourth part. The fifth part may have across-section within 80% of the cross-section of the adjacent sections of the surgical instrument. The first part may form less than 20% of the section of stem received in the intermediate component. The second part may form more than 50% of the section of stem received in the intermediate component. The third part may form less than 20% of the section of stem received in the intermediate component. The fourth part may provide less than 20% of the section of stem received in the intermediate component. The fifth part may not be received within the intermediate component.

In a third embodiment, particularly the section of stem received in the intermediate component, may provide a first part of the stem may be provided with a cross-section which increases away from the distal end. The first part may define an end for the stem. A second part of the stem may be provided with a cross-section which increases, for instance a slight taper. No third part of the stem may be provided in which the cross-section decreases further away from the distal end and then increases again still further away from the distal end. A third part of the stem may be provided intermediate the second part and a fourth part that is not received within the intermediate component. The fourth part may be provided with a larger cross-section than the first part and/or second part and/or third part. The fourth part may have across-section within 80% of the cross-section of the adjacent sections of the surgical instrument. The first part may form less than 20% of the section of stem received in the intermediate component. The second part may form more than 50% of the section of stem received in the intermediate component. The third part may form less than 20% of the section of stem received in the intermediate component. The fifth part may not be received within the intermediate component.

The intermediate component may be formed from one or more of stainless steel, tungsten carbide, titanium, ceramics or plastics. Potential ceramics may include aluminum oxide and calcium phosphates. Potential plastics materials may include ultra-high molecular weight polyethylene [UHMW], polycarbonates, acrylonitirile butadience styrene [ABS] and polydicyclopentadiene polymers [PDCPD]. The intermediate component may be provided with a coating, for instance covering the impact bearing surfaces.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The first aspect may include any of the features, possibilities or options set out elsewhere in this document, including in the other aspects.

According to a second aspect of the disclosure there is provided an intermediate component, potentially for use in a surgical component mounting system, the intermediate component having a proximal end, the proximal end of the intermediate component providing a second part of a mounting, potentially of a mounting for use in connecting to a surgical component, the intermediate component having a distal end, the distal end of the intermediate component being provided with a first part of a second mounting, potentially for use in connecting to a surgical implant, wherein the second part of the mounting and the first part of the second mounting are detachable mountings.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The second aspect may include any of the features, possibilities or options set out elsewhere in this document, including in the other aspects.

According to a third aspect of the disclosure there is provided a surgical instrument, particularly for use in a surgical component mounting system, the surgical instrument having a proximal end and a distal end, the distal end of the surgical instrument providing a first part of a mounting, wherein the first part of the mounting is a detachable mounting.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The third aspect may include any of the features, possibilities or options set out elsewhere in this document, including in the other aspects.

According to a fourth aspect of the disclosure there is provided a kit, the kit including:
at least one surgical instrument, the surgical instrument having a proximal end and a distal end, the distal end of the surgical instrument providing a first part of a mounting;
at least one intermediate component, the intermediate component having a proximal end, the proximal end of the intermediate component providing a second part of the mounting, the intermediate component having a distal end, the distal end of the intermediate component being provided with a first part of a second mounting; wherein the first part of the mounting and the second part of the mounting are detachable.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The fourth aspect may include any of the features, possibilities or options set out elsewhere in this document, including in the other aspects.

According to a fifth aspect of the invention there is provided a method of mounting a surgical instrument using a surgical component mounting system, the method including:
providing a surgical instrument, the surgical instrument having a proximal end and a distal end, the distal end of the surgical instrument providing a first part of a mounting;
providing an intermediate component, the intermediate component having a proximal end, the proximal end of the intermediate component providing a second part of the mounting, the intermediate component having a distal end, the distal end of the intermediate component being provided with a first part of a second mounting;
connecting he first part of the mounting and the second part of the mounting, the connection being detachable.

The method may include a detached state for the surgical instrument and the intermediate component in which the two are separate from one another. The method may include a step of bringing the distal end of the surgical instrument and the proximal end of the intermediate component into proximity, for instance to give the detached state.

The method may include a contacting state for the surgical instrument and the intermediate component. The method may include inserting a section of one of the surgical instrument and the intermediate component into the other of the intermediate component and the surgical instrument, for instance to give the contacting state. The method may include a section of the surgical instrument being inserted into the intermediate component, for instance to give the contacting state.

The method may include an engaged state. The method may include inserting the one of the surgical instrument and intermediate component into the other, for instance until an end face of the one abuts a base wall of the other. The method may include inserting the one of the surgical instrument and intermediate component into the other until no further insertion is possible, for instance to give an engaged state.

In the engaged state, the engagement may be provided between one or more retention elements on the surgical instrument and/or one or more retention elements on the intermediate component.

The method may include relative movement between the surgical instrument and the intermediate component until one or more retention elements on the surgical instrument and/or one or more retention elements on the intermediate component are brought into engagement, for instance to give an engaged state. For instance, a groove on one may engage with a rib on the other.

In the engaged state, the engagement may be provided by an interference fit between the surgical instrument and the intermediate component.

In the engaged state, axial movement of the surgical component and the intermediate component relative to one another may be restrained in one or both axial directions. The restraint may be sufficient to retain the intermediate component on the surgical instrument or vice versa. The restraint may be insufficient to stop a user pulling the intermediate component off the surgical instrument.

The method may include a second detached state for the surgical instrument and the intermediate component combination in which that combination is separate from a further surgical instrument and/or implant. The method may include a step of bringing the distal end of the intermediate component and the proximal end of the further surgical instrument and/or implant into proximity, for instance to give the second detached state.

The method may include a second contacting state for the surgical instrument and the intermediate component combination with a further surgical instrument and/or implant. The method may include inserting a section of one of the further surgical instrument and/or implant into the intermediate component, for instance to give the second contacting state. The method may include inserting a section of the intermediate component into the further surgical instrument and/or implant, for instance to give the second contacting state.

The method may include a second engaged state. The method may include inserting the intermediate component into the further surgical instrument and/or implant, for instance until an end of a projection of one abuts a base wall of the other. The method may include inserting the further surgical instrument and/or implant into the intermediate component, for instance until an end of a projection of one abuts a base wall of the other. The method may include inserting the one into the other until no further insertion is possible, for instance to give a second engaged state.

In the second engaged state, further axial movement of the surgical instrument and the intermediate component combination and the further surgical instrument and/or implant may be resisted. In the second engaged state, axial movement of the surgical instrument and the intermediate component combination away from the further surgical instrument and/or implant may not be resisted.

The method may further include moving and/or adjusting the position of the further surgical instrument and/or implant.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The fifth aspect may include any of the features, possibilities or options set out elsewhere in this document, including in the other aspects.

### BRIEF DESCRIPTION OF THE DRAWINGS

Various embodiments of the disclosure will now be described, by way of example only, and with reference to the accompanying figures, in which:

FIG. 1a shows a distal end of an impaction device adjacent a femoral component, without a cap;

FIG. 1b shows the distal end of the impaction device engaged with the femoral component in a femur;

FIG. 2a is an anterior or posterior view of a first embodiment cap according to the disclosure;

FIG. 2b is a medial or lateral view of the cap of FIG. 2a;

FIG. 2c is a partial inferior view of the cap of FIGS. 2a and 2b;

FIG. 3a is an anterior or posterior view of the distal end of an impaction device for use with the cap of FIGS. 2a, 2b and 2c;

FIG. 3b is an inferior view of the distal end of the impaction device of FIG. 3a;

FIG. 4a is an anterior or posterior view of a second embodiment cap according to the disclosure;

FIG. 4b is a medial or lateral view of the cap of FIG. 4a;

FIG. 5a is an anterior or posterior view of the distal end of an impaction device for use with the cap of FIGS. 4a and 4b;

FIG. 5b is an inferior view of the distal end of the impaction device of FIG. 3a;

FIG. 6a is an anterior or posterior view of a third embodiment cap according to the disclosure;

FIG. 6b is a medial or lateral view of the cap of FIG. 6a;

FIG. 7a is an anterior or posterior view of the distal end of an impaction device for use with the cap of FIGS. 7a and 7b;

FIG. 7b is an inferior view of the distal end of the impaction device of FIG. 3a;

FIG. 8a is an anterior or posterior view of a fourth embodiment cap according to the disclosure;

FIG. 8b is an inferior view of the cap of FIG. 8a;

FIG. 9a is an anterior or posterior view of the distal portion of an impaction device for use with the cap of FIGS. 8a and 8b;

FIG. 9b is an inferior view of the distal end of the impaction device of FIG. 9a;

FIG. 10a is an anterior or posterior view of a fifth embodiment cap according to the disclosure;

FIG. 10b is an inferior view of the cap of FIG. 10a;

FIG. 11a is an anterior or posterior view of the distal portion of an impaction device for use with the cap of FIGS. 10a and 10b;

FIG. 12a is an anterior or posterior view of a sixth embodiment cap according to the disclosure;

FIG. 12b is an inferior view of the cap of FIG. 12a;

FIG. 13a is an anterior or posterior view of the distal portion of an impaction device for use with the cap of FIGS. 12a and 12b;

FIG. 13b is an inferior view of the distal end of the impaction device of FIG. 12a; and

FIG. 14 references various typical parts for a cap according to an embodiment for guidance on dimensions.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

Impaction devices are used in a variety of surgical procedures, including in orthopaedic procedures, to apply force to another device or implant to insert or otherwise move that another device or implant. The proximal end of the impaction device is designed to have the impact force applied to it in use. The distal end of the impaction device is designed to abut, more often, engage with the proximal end of the another device or implant.

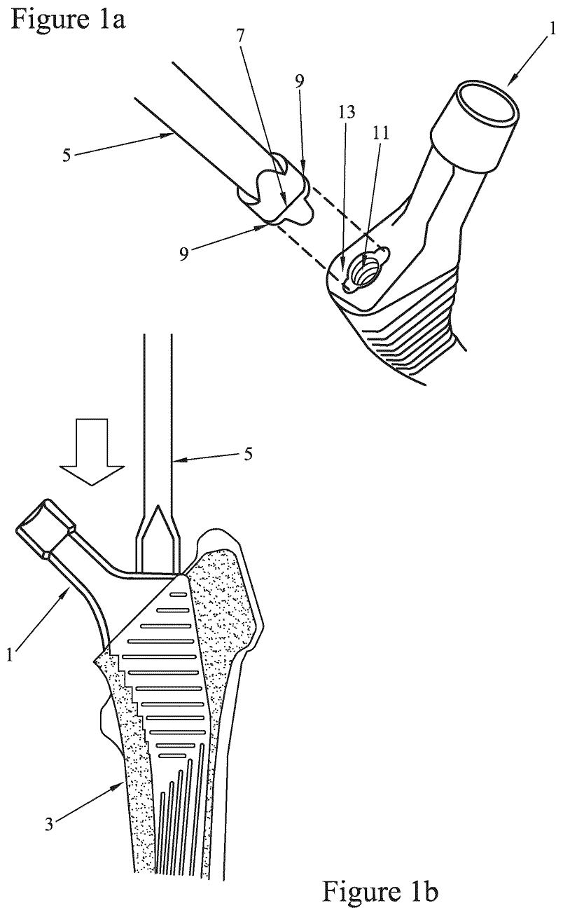

An example of such an approach in an orthopaedic procedure is shown in FIG. 1a and FIG. 1b in the context of a femoral implant 1 which is being moved relative to a femur 3. The impaction device 5 is provided at the distal end 7 with a pair of tines 9. The femoral implant 1 is provided with a complimentary recess 11. The impaction device 5 is maneuvered until the tines 9 align with the corresponding parts 13 of the complimentary recess 11 [FIG. 1a]. The impaction device 5 is then advanced towards the femoral implant 1 such that the distal end 7 of the impaction device 5 enters the complimentary recess 11. Once engaged in this manner [FIG. 1b] impaction forces can be applied to the proximal end [not shown] of the impaction device 5 and these are transmitted through the distal end 7 to the femoral implant 1.

It is desirable to provide an alternative engagement between the distal end of the impaction device and the another device or implant, for reasons that include: reducing the risk of impaction device [such as stem inserter tip] fracture during operation; and reducing deformation around the impaction seat on the implant.

In the applications of interest, material levels of impact force are applied to the impaction device and transmit through to the implant. Misalignment of the two, misconnection of the two or other factors can all lead to lateral components to the forces. These lateral components can, with repeated use, give rise to a risk of fatigue cracking in the interface at the distal end of the surgical instrument, even though the remainder of the impactor device has material lifetime left.

The approach described in the embodiments below allows for greater control of the number of times that the impaction device to implant interface is used to apply impact forces to the implant and hence to mitigate any risk of fatigue cracking of the surgical instrument. This is achieved by providing an intermediate component between the surgical instrument and the further surgical instrument or implant to which impact force is to be applied.

The approach described below is also beneficial as varying the intermediate component to further surgical instrument or implant mounting means that a common surgical instrument [such as a stem inserter shaft] can be used for a wider range of products; the intermediate component acts as an adaptor function.

The intermediate component is able to use existing engagements with the further surgical instrument and/or implants, but because after use, the intermediate component can be discarded, there is no risk of any fatigue cracking or the like.

The intermediate component enables more resilient interfaces to be provided between the surgical instrument and the intermediate component so that the surgical instrument is more resistant to any lateral force component and/or is not subject to fatigue.

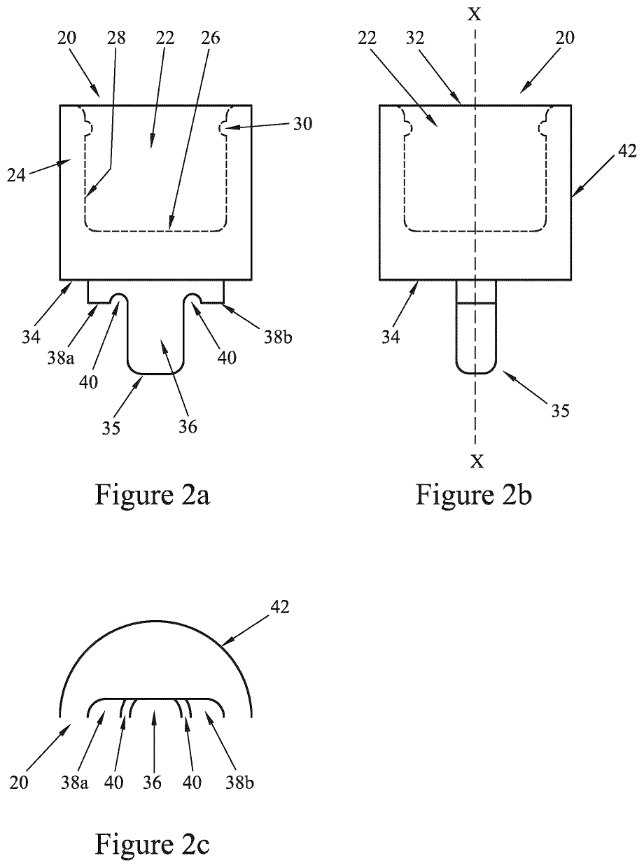

A cap element 20 style intermediate component, as illustrated in FIGS. 2a, 2b and 2c, may be provided for positioning between the distal end of the impaction device and the another device or implant, in use, particularly when impact forces are being transmitted to the another device or implant.

The cap element 20 has a bore 22 defined by a side wall 24, base wall 26 and a fillet 28 between those. The side wall 24 is provided with a rib 30 which extends around the internal perimeter of the bore 22. The rib 30 is provided at a constant depth relative to the mouth 32 of the bore 22 and at a constant height relative to the base wall 26 of the bore 22. The mouth 32 outwardly tapers to assist with introduction of the impaction device.

The inferior surface 34 of the cap element 20, and hence the distal end 35 of the cap element 20, is provided with a tip 36 and to either side of that tip 36 with a tine 38a, 38b separated from the tip 36 by a recess 40. The recess 40 is formed from two sections of an annulus. The tip 36 has a greater extent, medially-laterally [FIG. 2a] than anteriorly-posteriorly [FIG. 2b].

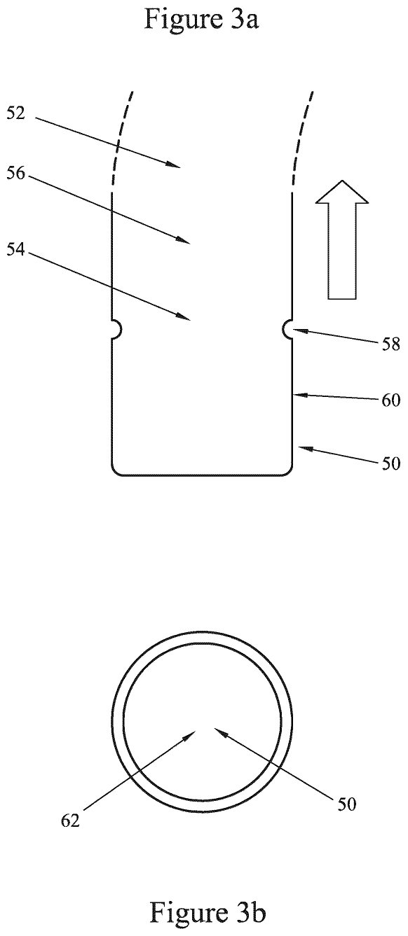

The external wall 42 of the cap element 20 is a right cylinder and may be provided with grooves and ridges or other recess/protrusion combinations to assist with grip. The bore 22 has a circular cross-section relative to the longitudinal axis X-X of the cap element 20. The bore 22 is adapted to receive part of the impactor device [FIGS. 3a and 3b]. The diameter of bore 22 can be readily configured during manufacture to render the cap element 20 compatible with a particular impaction device 52 from amongst a wide variety of impaction devices with different diameter distal ends 50.

In FIG. 3a, the distal end 50 of the impaction device 52 is shown. In this embodiment, a cylindrical end section 54 is provided at the distal end 50 and the longitudinal axis of this section may be off set relative to the longitudinal axis of an intermediate section 56, for instance to provide an offset to the impact part [not shown] at the proximal end of the impaction device 52.

A groove 58 is provided in the exterior surface 60 and this groove 58 extends around the entire circumference of the distal end 50 and is provided at a constant separation from the end surface 62 of the distal end 50.

The end section 54 is configured to be complimentary to the bore 22 in the cap element 20. Thus the end section 54 has: a radius which is a tolerance less than the radius of the bore 22; a chamfered edge 56 which is a tolerance less than the profile of the fillet 28; and a height for the groove 58 relative to the end surface 62 which is a tolerance less than the height of the rib 30 relative to the base wall 26 of the bore 22. The fillets and radii provided serve as stress reduction features.

In use, the cap element 20 and the distal end 50 of the impaction device are brought close to one another, a detached state, and then the distal end 50 is inserted into the bore 22. The insertion continues until the end surface 62 abuts the base wall 26 and no further insertion is possible, an inserted state. In this position, the rib 30 is received within the complimentary profile of the groove 58, an attached state. The cap element 20 and the impaction device 52 thus clip together. The cooperation of the rib 30 and groove 58 resists axial movement of the cap element 20 relative to the impaction device 52; the cap element 20 is releasably retained on the impaction device 52.

Alternative embodiments are possible in which the inserted state is provided without an attached state, such that there is no or little resistance to axial movement of the cap element 20 relative to the impaction device 52 away from one another again.

In this attached state, the distal end 35 of the cap element 20 can be aligned with and inserted into the another device or implant in a like manner to that described above in relation to FIG. 1a an 1b, an impactable state. To do so, the tip of the cap element 20 is brought into proximity with the another device or implant and then the tip 36 and tines 38a, 38b are aligned with a complimentary recess in the another device or implant and the tip 36 and tines 38a, 38b are then inserted into the complimentary recess. Once inserted a secure and stable interface is provided between the impaction device and the another device or implant and so impact forces can be successfully applied.

The cap element 20 can be versatile in its profile whilst still successfully integrating with the distal end 7 the impaction device 5 and so is suitable for adoption with a wide variety of impaction devices 5 with simple modification. In this way, the cap element 20 can be reconfigured for a different geometry of interface with the impaction device and/or different geometry of interface with the another device or implant. For instance, the depth of the bore 22 can be easily increased or decreased and/or the diameter can be reconfigured and/or the groove/recess combination can have a different profile without wholescale redesign of the cap element 20.

The cap element 20 is intended to be for a single surgical procedure use only. The impaction device 52 is intended for reuse in multiple procedures.

By being single use only, the cap element 20 is designed to significantly reduce the risk of distal tip fracture during surgery. As a single use cap element 20, it is possible to produce the cap element 20 from softer materials, than are used in the impaction device 5, and so reduce any deformation of further surgical instruments and/or implants it contacts [for instance the stem removal bore of an implant].

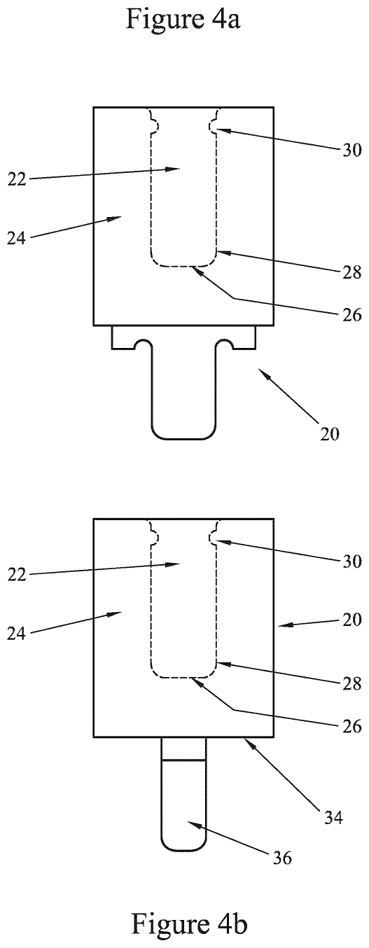

In FIGS. 4a and 4b an alternative embodiment for the cap element 20 is provided. In this case, an alternative “click together” format is provided. The cap element 20 again has a bore 22 defined by a side wall 24, base wall 26 and a fillet 28 between those. The side wall 24 is provided with a rib 30 which extends around the internal perimeter of the bore 22. Once again the rim is provided at a constant depth relative to the mouth 32 of the bore 22 and has a constant height relative to the base 26 and the bore 22.

Once again, the inferior surface 34 of the cap element 20 is provided with a tip 36 and tines 38a, 38b separated from the tip 36 by a recess 40.

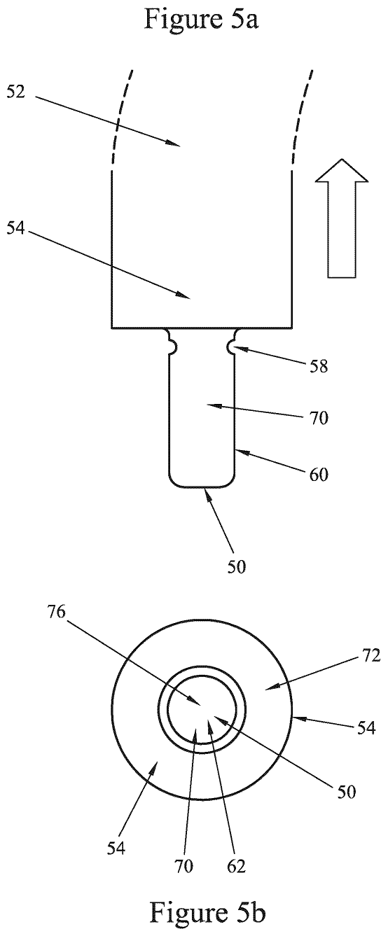

The bore 22 again has a circular cross section relative to the longitudinal axis X-X of the cap element 20. The bore 22 is adapted to receive part of the impactor device [FIGS. 5a and 5b]

As can be seen in FIG. 5a, the distal end 50 of the impaction device 52 is provided with a reduced diameter stem part 70 whose diameter is reduced when compared with the remainder of the distal end 50 of the impaction device 52. As before, the cylindrical end section 54 is provided and provides the mount for the stem part 70. The stem part 70 is provided with a groove 58 in its exterior surface 60, and this groove 58 extends around the entire circumference of the stem part 70. The groove is provided at a constant separation from the end surface 62 of the distal end 50 and also at a constant separation from the transition surface 72 at the end of the cylindrical end section 54.

The stem part 70 is configured to be complimentary to the bore 22 and the cap element 20. Thus the stem part 70 has: a radius which is a tolerance less than the radius of the bore 22; a chamfered edge 56 which is a tolerance less than the profile of the fillet 28; and the height for the groove 58 relative to the end surface 62 which is a tolerance less than the height of the rib 30 relative to the base wall 26 of the bore 22.

The stem part 70 tapers slightly from its proximal end 76 to the distal end 52. This exists with the introduction of the distal end 50 into the bore.

In use, the cap element 20 and the distal end 50 of the impaction device are brought close to one another and introduced to one another in the same manner as described above. Thus, the cap element 20 has a detached state, an inserted state in which the end surface 62 abuts the base wall 26 to prevent further insertion, and with the position surface 72 abutting the superior wall 74 of the cap element 20. Again, the rib 30 is received within the complimentary profile of the groove 58, the attached date.

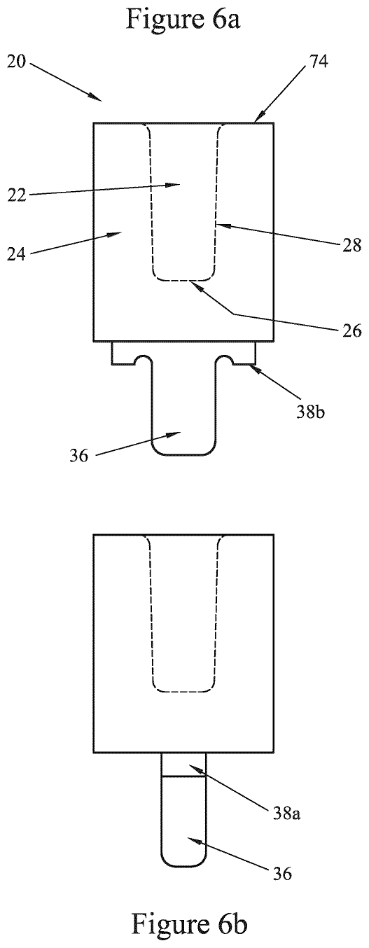

In FIGS. 6a and 6b, a further alternative embodiment for the cap element 20 is provided. In this case, an interference fit approach is employed. The cap element 20 has a bore 22 defined by a side wall 24, base wall 26 and a fillet 28 between those. The superior wall 74 is also provided. In this embodiment, no rib or groove arrangement is provided and the side wall 24 is continuous, but potentially tapering.

The tip 36, tines 38a, 38b ad recess 40 are provided in a similar manner to described above.

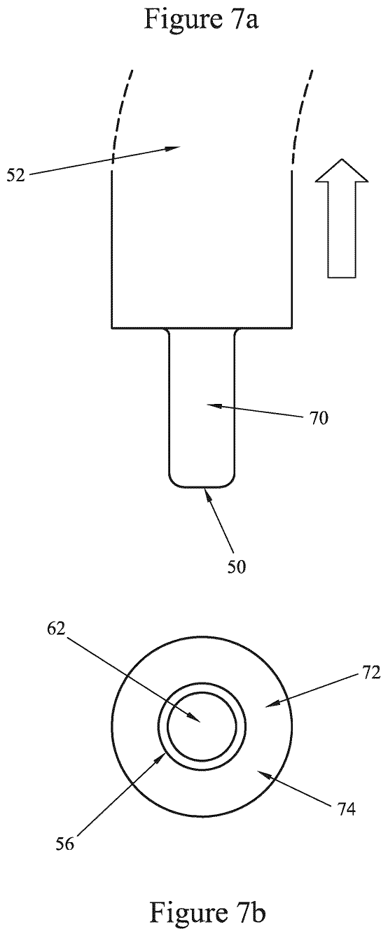

The stem part 70, at the distal end 50 of the impaction device 52, as shown in FIGS. 7a and 7b is also devoid of any recess or groove in this form.

The stem part 70 is configured to be complimentary to the bore 22 in a cap element. Thus, the stem part 70 has: a radius which is a tolerance less than the radius of the bore 22; a chamfered edge 56 which is a tolerance less than the profile of the fillet 28; and a length for the stem part 70 along the longitudinal axis X-X which is a tolerance less than the depth of the bore 22.

In use, the cap element 20 and the distal end 50 of the impaction device are brought together and insertion occurs. The insertion continues until the end surface 62 abuts the base bore 26 and the transition surface 72 abuts the superior wall 74 to prevent any further insertion. The interference fit provided between the stem part 70 and the bore 22 serves to retain the cap element 20 on the impaction device 5 during use.

Other embodiments of the engagement between the cap element 20 and the impaction device 52 are possible. For instance:

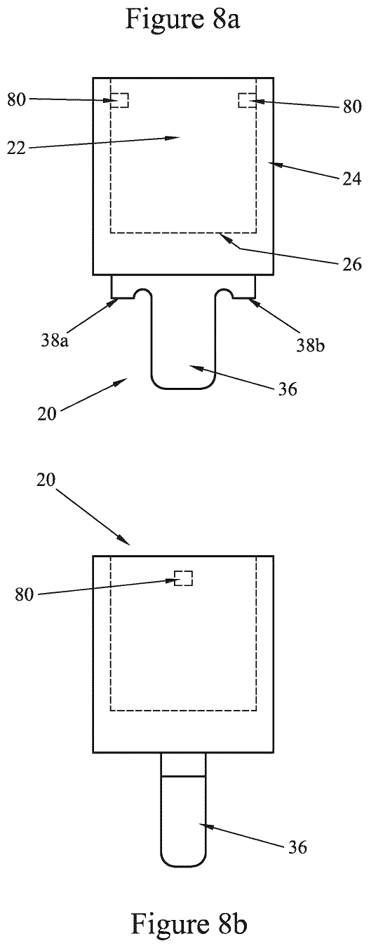

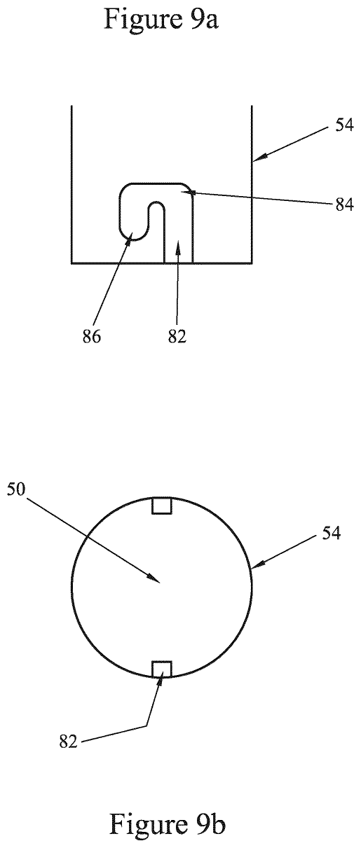

In FIGS. 8a and 8b, together with FIGS. 9a and 9b, a bayonet type engagement is employed. The cap element 20 has a bore 22, base wall 26 and side wall 24 as before, but the side wall 24 carries a plurality of lugs 80 which project inwardly. These can be inserted axially into the opening part 82 of a recess in the end section 54, then to the base part 84 of the recess and with rotation into the locking end part 86 of the recess.

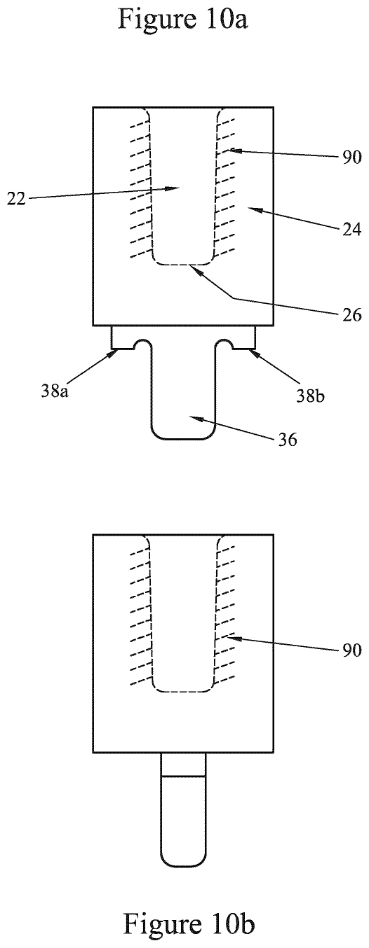

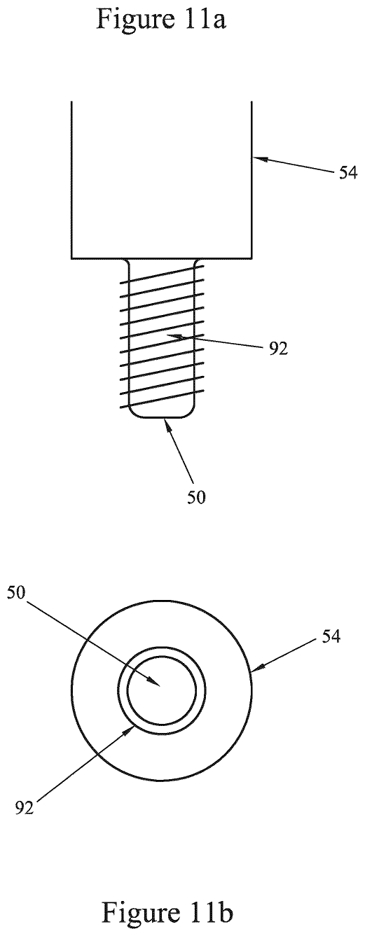

In FIGS. 10a and 10b, together with FIGS. 11a and 11b, a screw type engagement is provided. The cap element 20 has a bore 22, base wall 26 and side wall 24 as before, but the side wall 24 are provided with a screw thread 90. After initial insertion, rotation of the cap element 20 with causes the engagement and increasing threaded engagement with the impaction device and its thread 92.

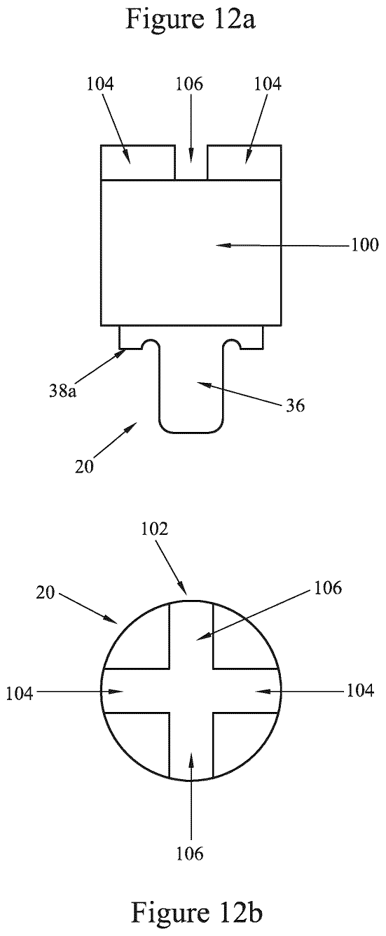

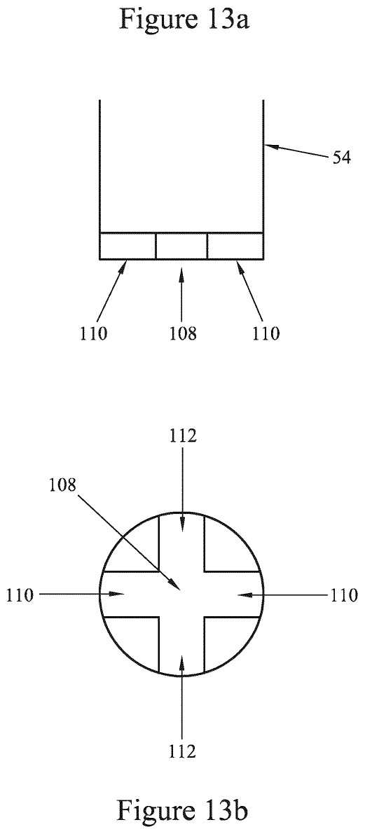

In FIGS. 12a and 12b, together with FIGS. 13a and 13b a complimentary surface type engagement is provided. The cap element 20 is provided with a solid body 100 with a cross-shaped recess 102 formed in the proximal end of the solid body 100. The cross-shaped recess 102 is formed by two intersecting channels 104 and 106 provided in this case at right angles to each other. The distal end of the impaction device is provided with a cross-shaped projection 108 which is complimentary n profile to the cross-shaped recess 102. The cross-shaped projection is formed by two intersecting protrusions 110 and 112.

In terms of suitable materials for the cap element 20, those materials need to be able to successfully withstand the worst likely case scenario for a single use successfully. Any change in the cap element 20 should be below a level which impacts upon the efficacy of the system. Plastics, metals and ceramics all offer potential materials for the cap element 20.

A single use of the cap element 20 may be ensured by careful selection of the materials for the cap element 20 relative to conditions that would be encountered in any attempt to re-use the cap element 20. For instance, the materials may be selected to be destroyed or at least rendered inoperative by washing and/or autoclaving of the cap element 20.

A wide range of materials are suitable for use to form the cap element 20. The cap element 20 could be formed from the same material or material type as the surgical instrument, for example impaction device 52, it is to be mounted on. Suitable materials include stainless steel, tungsten carbide, titanium, selected ceramics and selected plastics. Potential ceramics include aluminum oxide and calcium phosphates. Potential plastics include polyphenylsulfones [such as Radel® of Solvay], polyetherketones [PEEK] and polypropylene resins [such as Propylux® available from Westlake Plastics]. Further potential plastics materials could include ultra-high molecular weight polyethylene [UHMW], polycarbonates, acrylonitirile butadience styrene [ABS] and polydicyclopentadiene polymers [PDCPD].

The cap element 20 may be provided with a partial or full coating, for instance a partial ceramic coating covering the impact bearing surfaces, to modify the properties at a location on the cap element 20.

As the cap element 20 is intended for single use, a wider range of materials are potentially suitable than for repeatedly used surgical instruments. As a single use component, the cap element 20 may be provided from less expensive materials than the surgical instruments.

Whilst intended for reuse and then discard, ideally for recycling of the material, it is desirable to actively prevent inadvertent reuse of the cap element 20. There are various approaches for achieving or contributing to this.

Potential approaches include a visible change indicator, if an attempt to prepare the cap element 20 for reuse occurs. An attempt to prepare the cap element 20 for reuse would include the likes of: cleaning/washing; thermal disinfection [typically at 73° C. to 90° C.] and/or chemical disinfection; sterilization [typically saturated steam at 130° C. to 137° C. for 3 to 3.5 mins]. The visible change indicator would respond to exposure to one or more of those steps and the conditions they represent, by undergoing a visible change in the appearance of the visible change indicator and hence flag prior use having occurred or that the cap was at risk of reuse.

Potential approaches include a physical change in one or more features of the cap element 20, as a result of the preparations for reuse. Deformation of one or more parts of the cap element 20 would be one possibility. For instance, the temperature encountered could result in the retention element of the cap element 20 [for instance the rib 30 or the internal profile of the bore 22 which provides the interference fit] altering in physical format or profile such that they no longer provided for mounting of the cap element 20 on the impaction device 52. The retention element, such as the rib 30, is not used in the transmission of impact forces through the cap element 20 and is merely used to hold the two together during maneuvering into the position of use. Thus, the retention element could be formed of a material which undergoes such a physical transformation under such conditions readily, as a wide range of materials are suitable for providing such a low of retention to just hold the cap element 20 on the impaction device 52. For instance, polyethylene oxide [PEG] melts at around 66° C.

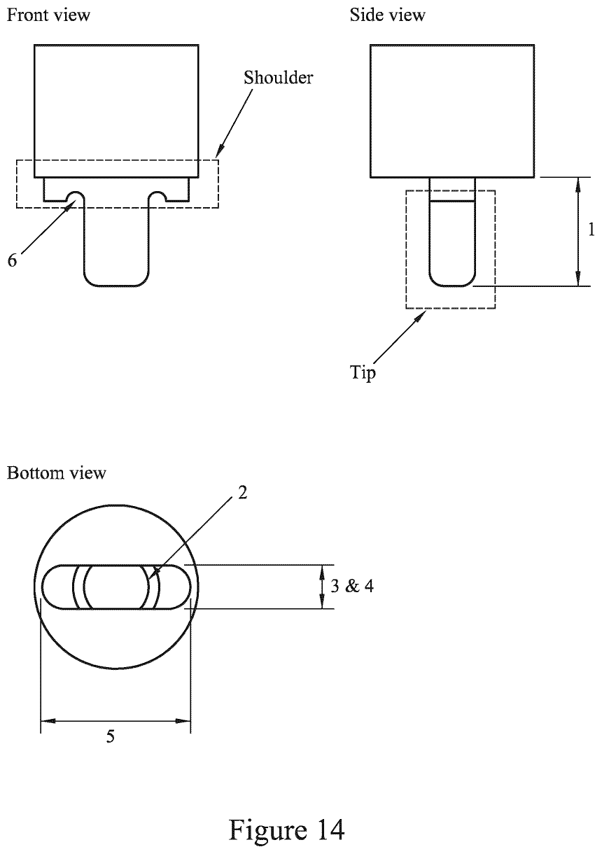

Referring to FIG. 14, a series of parts are marked and the following represent exemplary only embodiments for the dimensions for those parts.
Part 1 dimension—Inferior surface 34 to end of tip 36=7.870±0.125 mm to 14.870±0.125 mm;
Part 2 dimension—Diameter or long axis length of tip 36=4.615±0.050 mm to 4.917_{-0.022}^{-0.010} mm
Part 3 dimension—Width or short axis length of tip 36=3.650±0.050 mm to 3.900_{-0.022}^{-0.010}
Part 4 dimension—Width or short axis length of tine 38a and/or tine 38b [for instance anterior/posterior extent]=3.650±0.050 mm to 3.900_{-0.022}^{-0.010} mm
Part 5 dimension—Diameter or long axis length of tine 38a to 38b [for instance medial/lateral extent]=10.600±0.125 mm to 11.000_{-0.022}^{-0.010} mm
Part 6—Provision of a radius is desirable between the tip and shoulder.

Whilst the embodiments described above focus on the cap element 20 in the context of an impaction device 52, the concept of the cap element 20 is applicable in other surgical instrument contexts where impact or other high and/or varying force levels need to be applied. Further examples of such a scenario could include an acetabular shell inserter, acetabular poly liner inserter, acetabular drill holder and the like.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A surgical component mounting system, the system comprising:
a surgical instrument, the surgical instrument having a proximal end and a distal end, the distal end of the surgical instrument providing a first part of a mounting;
an intermediate component, the intermediate component having a proximal end, the proximal end of the intermediate component providing a second part of the mounting, the intermediate component having a distal end, the distal end of the intermediate component being provided with a first part of a second mounting; wherein the first part of the mounting and the second part of the mounting are detachable.

2. A surgical component mounting system according to claim 1, wherein the intermediate component is a single surgical occasion use component and the intermediate component is provided with one or more characteristics which inhibit the intermediate component being a multiple surgical occasion use component.

3. A surgical component mounting system according to claim 2, wherein the one or more characteristics are characteristics susceptible to change under one or more conditions and the one or more conditions are one or more of:
one or more conditions experienced by the intermediate component if prepared for reuse;
a washing temperature for the intermediate component;
exposure of the intermediate component to one or more chemicals;
exposure of the intermediate component to a disinfecting temperature;
exposure of the intermediate component to a sterilising temperature.

4. A surgical component mounting system according claim 2, wherein the one or more characteristics are one or more visual indicia.

5. A surgical component mounting system according to claim 2, wherein the one or more characteristics are a changeable part of the intermediate component and the one or more characteristics are a part of the intermediate component that changes profile and/or shape and/or that ceases to be present and/or that ceases to function.

6. A surgical component mounting system according to claim 2, wherein the detachable mounting is adapted to, in an engaged state, restrain axial movement of the surgical component and the intermediate component relative to one another in one or both axial directions.

7. A surgical component mounting system according to claim 2, wherein the intermediate component is a cap element.

8. A surgical component mounting system according to claim 2, wherein the intermediate component provides a body part, a recess in the body part and wherein the recess is provided with one or more retention elements.

9. A surgical component mounting system according to claim 8, wherein the recess is provided with one or more projections extending inwardly.

10. A surgical component mounting system according to claim 9, wherein the distal end of the intermediate element provides a first part of the second mounting which includes one or more projections which extend in a distal direction

11. A surgical component mounting system according to claim 10, wherein the first part of the second mounting include a primary projection and one or two or more secondary projections, wherein the primary projection is provided between the two or more secondary projections.

12. A surgical component mounting system according to claim 11, wherein the surgical instrument is an orthopaedic instrument and/or wherein the surgical instrument is an impactor instrument used to apply axial force or substantially axial force.

13. A surgical component mounting system according to claim 12, wherein the intermediate component is connectable to an implant and/or a femoral implant and/or an acetabular cup and/or an acetabular shell.

14. A method of mounting a surgical instrument using a surgical component mounting system, the method including:
providing a surgical instrument, the surgical instrument having a proximal end and a distal end, the distal end of the surgical instrument providing a first part of a mounting;
providing an intermediate component, the intermediate component having a proximal end, the proximal end of the intermediate component providing a second part of the mounting, the intermediate component having a distal end, the distal end of the intermediate component being provided with a first part of a second mounting;
connecting he first part of the mounting and the second part of the mounting, the connection being detachable.

15. The method of claim 14, wherein, the method includes a number of states, the number of states including:
a detached state for the surgical instrument and the intermediate component in which the two are separate from one another;
a contacting state for the surgical instrument and the intermediate component in which a section of one of the surgical instrument and the intermediate component is inserted into the other of the intermediate component and the surgical instrument; and
an engaged state including inserting the one of the surgical instrument and intermediate component into the other until no further insertion is possible.

16. The method of claim 15, wherein in the engaged state:
the engagement is provided between one or more retention elements on the surgical instrument and/or one or more retention elements on the intermediate component; and/or
the engagement is provided by an interference fit between the surgical instrument and the intermediate component; and/or
axial movement of the surgical component and the intermediate component relative to one another are restrained in one or both axial directions.

17. The method of claim 14, wherein the method provides:
a second detached state for the surgical instrument and the intermediate component combination, in which that combination is separate from a further surgical instrument and/or implant; and/or
a second contacting state for the surgical instrument and the intermediate component combination with a further surgical instrument and/or implant.
